SEQUENCE LISTING (2 sequences)

SEQ ID NO: 1 - DNA, 9234 bp, Homo sapiens
  gene (1)...(9234): Homo sapiens leucine-rich repeat kinase 2 (LRRK2), mRNA (NM_198578)
cgctggctgcgggcggtgagctgagctcgcccccggggagctgtggccggcgcccctgccggttccctgagcagcggacgttcatgctgggagggcggcgggttggaagcaggtgccaccatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcacgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatattggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgtcaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggcattatttcgcgtagagatgtggaaaaatttctttcaaaaaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaaggtacacaaaagcagaaagagatacaatcttgcttgaccgtttgggacatcaatcttccacatgaagtgcaaaatttagaaaaacacattgaagtgagaaaagaattagctgaaaaaatgagacgaacatctgttgagtaagagagaaataggaattgtctttggataggaaaattattctctcctcttgtaaatatttattttaaaaatgttcacatggaaagggtactcacattttttgaaatagctcgtgtgtatgaaggaatgttattatttttaatttaaatatatgtaaaaatacttaccagtaaatgtgtattttaaagaactatttaaaacacaatgttatatttcttataaataccagttactttcgttcattaattaatgaaaataaatctgtgaagtacctaatttaagtactcatactaaaatttataaggccgataattttttgttttcttgtctgtaatggaggtaaactttattttaaattctgtgcttaagacaggactattgcttgtcgatttttctagaaatctgcacggtataatgaaaatattaagacagtttcccatgtaatgtattccttcttagattgcatcgaaatgcactatcatatatgcttgtaaatattcaaatgaatttgcactaataaagtcctttgttggtatgtgaattctctttgttgctgttgcaaacagtgcatcttacacaacttcactcaattcaaaagaaaactccattaaaagtactaatgaaaaaacatgacatactgtcaaagtcctcatatctaggaaagacacagaaactctctttgtcacagaaactctctgtgtctttcctagacataatagagttgtttttcaactctatgtttgaatgtggataccctgaattttgtataattagtgtaaatacagtgttcagtccttcaagtgatatttttatttttttattcataccactagctacttgttttctaatctgcttcattctaatgcttatattcatcttttccctaaatttgtgatgctgcagatcctacatcattcagatagaaacctttttttttttcagaattatagaattccacagctcctaccaagaccatgaggataaatatctaacacttttcagttgctgaaggagaaaggagctttagttatgatggataaaaatatctgccaccctaggcttccaaattatacttaaattgtttacatagcttaccacaataggagtatcagggccaaatacctatgtaataatttgaggtcatttctgctttaggaaaagtactttcggtaaattctttggccctgaccagtattcattatttcagataattccctgtgataggacaactagtacatttaatattctcagaacttatggcattttactatgtgaaaactttaaatttatttatattaagggtaatcaaattcttaaagatgaaagattttctgtattttaaaggaagctatgctttaacttgttatgtaattaacaaaaaaatcatatataatagagctctttgttccagtgttatctctttcattgttactttgtatttgcaattttttttaccaaagacaaattaaaaaaatgaataccatatttaaatggaataataaaggttttttaaaaactttaaaaaaaaaaaaa

SEQ ID NO: 2 - protein, 2527 aa, Homo sapiens
  PROPEP (1)...(2527): Homo sapiens leucine-rich repeat kinase 2 (LRRK2) preprotein (NM_198578)
MASGSCQGCEEDEETLKKLIVRLNNVQEGKQIETLVQILEDLLVFTYSEHASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSASTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

(19) United States
(12) Patent Application Publication
(10) Pub. No.: US 2010/0035251 A1
(43) Pub. Date: Feb. 11, 2010

(54) BioMarkers for the Progression of Alzheimer's Disease

(76) Inventors: Yunsheng He, Waltham, MA (US); Baltazar Gomez-Mancilla, Portage, MI (US); Joanne Meyer, Framingham, MA (US); Giorgio Rovelli, Arlesheim (CH); Graeme Bilbe, Neuchatel (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/305,053

(22) PCT Filed: Jun. 18, 2007
(86) PCT No.: PCT/US07/71421

§ 371 Date: Dec. 16, 2008

Related U.S. Application Data
(60) Provisional application No. 60/815,610, filed on Jun. 20, 2006.

(51) Int. Cl. C12Q 1/68; C07D 209/04

(52) U.S. Cl. 435/6; 548/468

(57) ABSTRACT

The genetic polymorphism LRRK2 (leucine-rich repeat kinase 2)-T1602S is significantly associated with conversion from mild cognitive impairment (MCI) to Alzheimer's disease (AD), with the patients with TT genotype being at greater risk to progress to Alzheimer's disease. The LRRK2-T2352 also showed a trend for conversion to Alzheimer's disease, with the patients with CC genotype tending to progress to Alzheimer's disease. Similar to the APOE-E4 allele, in the presence of a BuChE-K variant, LRRK2-T1602S and LRRK2-T2352 showed a greater association with the rate of conversion from mild cognitive impairment to Alzheimer's disease. In another study with placebo-treated Alzheimer's disease patients, LRRK2-T1602S and LRRK2-T2352 showed a same trend of association. The Alzheimer's disease patients with TT genotype of LRRK2-T1602S or CC genotype of LRRK2-T2352 tended to decline faster on cognitive performance over 6 months, especially in the presence of a BuChE-K variant. The association between the two common LRRK2 polymorphisms and Alzheimer's disease progression shows that LRRK2 may play a role in Alzheimer's disease pathogenesis, especially disease progression, and that polymorphisms of LRRK2 can be used as biomrkers of this progression.

### FIELD OF THE INVENTION

This invention relates generally to the analytical testing of tissue samples in vitro, and more particularly to aspects of genetic polymorphisms of the leucine-rich repeat kinase 2 (LRRK2) gene.

### BACKGROUND OF THE INVENTION

Therapy specific diagnostics (a.k.a., theranostics) is an emerging medical technology field, which provides tests useful to diagnose a disease, choose the correct treatment regime and monitor a subject's response. That is, theranostics are useful to predict and assess drug response in individual subjects, i.e., individualized medicine. Theranostic tests are also useful to select subjects for treatments that are particularly likely to benefit from the treatment or to provide an early and objective indication of treatment efficacy in individual subjects, so that the treatment can be altered with a minimum of delay.

Progress in pharmacogenetics, which establishes correlations between responses to specific drugs and the genetic profile of individual patients, is foundational to the development of new theranostic approaches. As such, there is a need in the art for the evaluation of patient-to-patient variations in gene sequence and gene expression. A common form of genetic profiling relies on the identification of DNA sequence variations called single nucleotide polymorphisms (“SNPs”), which are one type of genetic mutation leading to patient-to-patient variation in individual drug response. It follows that there is a need in the art to identify and characterize genetic mutations, such as SNPs, which are useful to identify the genotypes of subjects associated with drug responsiveness, side-effects, or optimal dose.

### SUMMARY OF THE INVENTION

Polymorphisms of the of the leucine-rich repeat kinase 2 (LRRK2) gene can be used as biomarkers of Alzheimer's disease (AD) progression. In particular, mutations of the LRRK2 gene causing a change in the protein (such as T1602S and T2352, in particular T2352M) may have an impact on Alzheimer's disease and can be used as a biomarker of Alzheimer's disease progression and age-at-onset of Alzheimer's disease. Accordingly, the invention provides for the use of a LRRK2 modulating agent in the manufacture of a medicament for the treatment of Alzheimer's disease a selected patient population. The patient population is selected on the basis of polymorphisms in the LRRK2 gene that are indicative of progression from mild cognitive impairment (MCI) to Alzheimer's disease. In one embodiment, the LRRK2 modulating agent is a heterocyclic compound that slows the progression by the patient from mild cognitive impairment to Alzheimer's disease. In another embodiment, the LRRK2 modulating agent is a heterocyclic compound that slows the progression by the patient from moderate Alzheimer's disease to severe Alzheimer's disease. In yet another embodiment, the polymorphism in the LRRK2 gene can be T1602S or T2352. The invention also provides methods for the predicting Alzheimer's disease progression or age-at-onset of Alzheimer's disease.

### BRIEF DESCRIPTION OF THE DRAWINGS

The drawing figure depicts preferred embodiments by way of example, not by way of limitations.

FIG. 1 is a depiction of the LRRK2 protein structure and location of the two common LRRK2 polymorphisms (T1602S and T2352M).

### DETAILED DESCRIPTION OF THE INVENTION

Progression to Alzheimer's disease data of a 3-4 year study in mild cognitive impairment (MCI) patients was used to investigate the effect of the two common LRRK2 polymorphisms, T1602S and T2352, on rate of progression to Alzheimer's disease. To verify the findings, we further tested the correlation between the two common LRRK2 polymorphisms and cognitive performance over 6 months in placebo-treated Alzheimer's disease patients enrolled in another study.

We found that LRRK2-T1602S was significantly associated with conversion from mild cognitive impairment to Alzheimer's disease. The mild cognitive impairment patients with TT genotype were at greater risk to progress to Alzheimer's disease. The LRRK2-T2352 also showed a trend for conversion to Alzheimer's disease. The mild cognitive impairment patients with CC genotype tended to progress to Alzheimer's disease. Similar to the APOE-E4 allele, in the presence of a BuChE-K variant, LRRK2-T1602S and LRRK2-T2352 showed a greater association with the rate of conversion from mild cognitive impairment to AD.

In the study with the placebo-treated Alzheimer's disease patients, LRRK2-T1602S and LRRK2-T2352 showed a same trend of the association observed in the mild cognitive impairment study. The Alzheimer's disease patients with TT genotype of LRRK2-T1602S or CC genotype of LRRK2-T2352 tended to decline faster on cognitive performance over 6 months, especially in the presence of a BuChE-K variant.

The association between the two common LRRK2 polymorphisms and Alzheimer's disease progression shows that LRRK2 may play a role in Alzheimer's disease pathogenesis, especially disease progression and that polymorphisms of LRRK2 can be used as biomrkers of this progression.

The human LRKK2 gene (SEQ ID NO:1) is located in the PARK8 locus on chromosome 12q12. The gene has multiple-domains. LRKK2 protein (SEQ ID NO:2)is a receptor interacting protein (RIP) kinase. The G2019S mutation is the most common pathogenic mutation of LRRK2 (5-6% of autosomal dominant and ˜1% of sporadic late-onset cases). The G2019S mutation is located in the kinase domain of the LRKK2 gene.

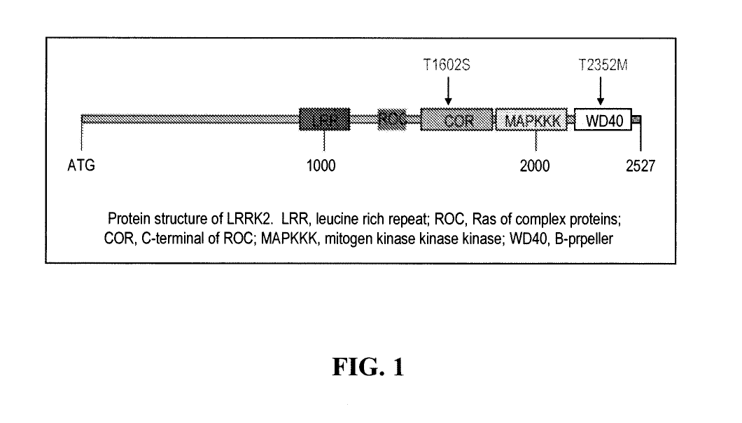

FIG. 1 shows LRRK2 protein structure and location of two other common polymorphisms. We focused on the polymorphisms T1602S and T2352M, because (1) they are common polymorphisms; and (2) they are missense polymorphisms.

The minor allele frequency is as follows: T1602S=30%; T2352M=34%. Amino acid change caused by the polymorphisms are as follows: T1602S-Thr→Ser (1602 A>T); T2352M-Thr→Met (2352 C>T). According to linkage disequilibrium (LD) analysis, T1602S and T2352M are in strong LD (D′=0.979).

It is to be appreciated that certain aspects, modes, embodiments, variations and features of the invention are described below in various levels of detail in order to provide a substantial understanding of the present invention. The various aspects of the present invention relate to diagnostic/theranostic methods and kits that use the LRRK2 mutations of the invention to identify individuals predisposed to disease or to classify individuals with regard to drug responsiveness, side effects, or optimal drug dose. In other aspects, the invention provides methods for compound validation and a computer system for storing and analyzing data related to the LRRK2 mutations of the invention. Accordingly, various particular embodiments that illustrate these aspects follow.

Definitions. The definitions of certain terms as used in this specification are provided below. Definitions of other terms may be found in the glossary provided by the U.S. Department of Energy, Office of Science, Human Genome Project (http: www.ornl.gov/sci/techresources/Human_Genome/glossary/). In practicing the present invention, many conventional techniques in molecular biology, microbiology and recombinant DNA are used. These techniques are well-known and are explained in, e.g., Current Protocols in Molecular Biology, Vols. I-III, Ausubel, ed. (1997); Sambrook et al., Molecular Cloning: A Laboratory Manual, Second Edition (Cold Spring Harbor Laboratory Press, Cold Spring Harbor, N.Y., 1989); DNA Cloning: A Practical Approach, Vols. I and II, Glover D, ed. (1985); Oligonucleotide Synthesis, Gait, ed. (1984); Nucleic Acid Hybridization, Hames & Higgins, eds. (1985); Transcription and Translation, Hames & Higgins, eds. (1984); Animal Cell Culture, Freshney, ed. (1986); Immobilized Cells and Enzymes (IRL Press, 1986); Perbal, A Practical Guide to Molecular Cloning; the series, Methods in Enzymol. (Academic Press, Inc., 1984); Gene Transfer Vectors for Mammalian Cells, Miller & Calos, eds. (Cold Spring Harbor Laboratory, N.Y., 1987); and Methods in Enzymology, Vols. 154 and 155, Wu & Grossman, and Wu, eds., respectively.

As used herein, the term “allele” means a particular form of a gene or DNA sequence at a specific chromosomal location (locus).

As used herein, the term “antibody” includes, but is not limited to, polyclonal antibodies, monoclonal antibodies, humanized or chimeric antibodies and biologically functional antibody fragments sufficient for binding of the antibody fragment to the protein. Antibodies can be used in assays to determine the presence of variant proteins and peptides where the genetic polymorphisms of the invention are in the coding region of the gene.

As used herein, the term “clinical response” means any or all of the following: a quantitative measure of the response, no response, and adverse response (i.e., side effects).

As used herein, the term “clinical trial” means any research study designed to collect clinical data on responses to a particular treatment, and includes but is not limited to phase I, phase II and phase III clinical trials. Standard methods are used to define the patient population and to enrol subjects.

As used herein, the term “effective amount” of a compound is a quantity sufficient to achieve a desired therapeutic and/or prophylactic effect, for example, an amount which results in the prevention of or a decrease in the symptoms associated with a disease that is being treated, e.g., the diseases associated with LRRK2 mutant polynucleotides and mutant polypeptides identified herein (particularly Alzheimer's disease and Parkinson's disease). The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the compounds of the present invention, sufficient for achieving a therapeutic or prophylactic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. The compounds of the present invention can also be administered in combination with each other, or with one or more additional therapeutic compounds.

As used herein, “expression” includes but is not limited to one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, the term “genotype” means an unphased 5′ to 3′ sequence of nucleotide pairs found at one or more polymorphic sites in a locus on a pair of homologous chromosomes in an individual. As used herein, genotype includes a full-genotype and/or a sub-genotype.

As used herein, the term “locus” means a location on a chromosome or DNA molecule corresponding to a gene or a physical or phenotypic feature, in particular the LRRK2 gene.

As used herein, the term “LRRK2 modulating agent” is any compound that alters (e.g., increases or decreases) the expression level or biological activity level of LRRK2 polypeptide compared to the expression level or biological activity level of LRRK2 polypeptide in the absence of the LRRK2 modulating agent. LRRK2 modulating agent can be a small molecule, polypeptide, carbohydrate, lipid, nucleotide, or combination thereof The LRRK2 modulating agent may be an organic compound or an inorganic compound.

As used herein, the term “mutant” means any heritable variation from the wild-type that is the result of a mutation, e.g., single nucleotide polymorphism. The term “mutant” is used interchangeably with the terms “marker”, “biomarker”, and “target” throughout the specification.

As used herein, the term “medical condition” includes, but is not limited to, any condition or disease manifested as one or more physical and/or psychological symptoms for which treatment is desirable, and includes previously and newly identified diseases and other disorders.

As used herein, the term “nucleotide pair” means the nucleotides found at a polymorphic site on the two copies of a chromosome from an individual.

As used herein, the term “polymorphic site” means a position within a locus at which at least two alternative sequences are found in a population, the most frequent of which has a frequency of no more than 99%.

As used herein, the term “phased” means, when applied to a sequence of nucleotide pairs for two or more polymorphic sites in a locus, the combination of nucleotides present at those polymorphic sites on a single copy of the locus is known.

As used herein, the term “polymorphism” means any sequence variant present at a frequency of >1% in a population. The sequence variant may be present at a frequency significantly greater than 1% such as 5% or 10% or more. Also, the term may be used to refer to the sequence variation observed in an individual at a polymorphic site. Polymorphisms include nucleotide substitutions, insertions, deletions and microsatellites and may, but need not, result in detectable differences in gene expression or protein function.

As used herein, the term “polynucleotide” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the term “polypeptide” means any polypeptide comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well-known in the art. Such modifications are well described in basic texts and in more detailed monographs, as well as in a voluminous research literature.

As used herein, the term “SNP nucleic acid” means a nucleic acid sequence, which comprises a nucleotide that is variable within an otherwise identical nucleotide sequence between individuals or groups of individuals, thus existing as alleles. Such SNP nucleic acids are preferably from about 15 to about 500 nucleotides in length. The SNP nucleic acids may be part of a chromosome, or they may be an exact copy of a part of a chromosome, e.g., by amplification of such a part of a chromosome through PCR or through cloning. The SNP nucleic acids are referred to hereafter simply as “SNPs”. A SNP is the occurrence of nucleotide variability at a single position in the genome, in which two alternative bases occur at appreciable frequency (i.e., >1%) in the human population. A SNP may occur within a gene or within intergenic regions of the genome. SNP probes according to the invention are oligonucleotides that are complementary to a SNP nucleic acid.

As used herein, the term “subject” means that preferably the subject is a mammal, such as a human, but can also be an animal, e.g., domestic animals (e.g., dogs, cats and the like), farm animals (e.g., cows, sheep, pigs, horses and the like) and laboratory animals (e.g., monkey (e.g., cynmologous monkey, rats, mice, guinea pigs and the like).

As used herein, the administration of an agent or drug to a subject or patient includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

LRRK2 Modulating Agents. In one embodiment, the LRRK2 modulating agent can be a hetrocyclic compound inhibitor of LRRK2 protein (SEQ ID NO:2).

In several embodiments, the heterocyclic compound can be 5-[5-Methoxy-2-oxo-1,2-dihydro-indol-(3Z)-ylidenemethyl]-2,4-dimethyl-1H-pyrrole-3-carboxylic acid (3-amino-propyl)-amide; 5-[6-Methoxy-2-oxo-1,2-dihydro-indol-(3Z)-ylidenemethyl]-2,4-dimethyl-1H-pyrrole-3-carboxylic acid (3-amino-propyl)-amide; 5-[7-Methoxy-2-oxo-1,2-dihydro-indol-(3Z)-ylidenemethyl]-2,4-dimethyl-1H-pyrrole-3-carboxylic acid (3-amino-propyl)-amide; 5-[5-Methoxy-2-oxo-1,2-dihydro-indol-(3Z)-ylidenemethyl]-2,4-dimethyl-1H-pyrrole-3-carboxylic acid (2-diethylamino-ethyl)-amide; or 5-[5--Dimethylsulfamoyl-2-oxo-1,2-dihydro-indol-(3Z)-ylidenemethyl]-2,4-dimethyl-1H-pyrrole-3-carboxylic acid (2-diethylamino-ethyl)-amide.

In other embodiments, the heteroccyclic compound can be 3-[1-(3,5-Dimethyl-1H-pyrrol-2-yl)-methyl-(Z)-ylidene]-5-methoxy-1,3-dihydro-indol-2-one; 3-[1-(1H-Indol-2-yl)-meth-(Z)-ylidene]-5-methoxy-1,3-dihydro-indol-2-one; 5-Methoxy-3-[1-(4,5,6,7-tetrahydro-1H-indol-2-yl)-meth-(Z)-ylidene]-1,3-dihydro-indol-2-one; 3-[1-(3,5-Dimethyl-1H-pyrrol-2-yl)-meth-(Z)-ylidene]-5-methoxy-2-oxo-2,3-dihydro-indol-4-carboxylic acid methyl ester; 3-[1-(3,5-Dimethyl-1H-pyrrol-2-yl)-meth-(Z)-ylidene]-5-methoxy-2-oxo-2,3-dihydro-indol-4-carboxylic acid ethyl ester; 3-[1-(3,5-Dimethyl-1H-pyrrol-2-yl)-meth-(Z)-ylidene]-5-methoxy-2-oxo-2,3-dihydro-indol-4-carboxylic acid methyl ester; or 3-[1-(3,5-Dimethyl-1H-pyrrol-2-yl)-meth-(Z)-ylidene]-5-methoxy-2-oxo-2,3-dihydro-indol-4-carboxylic acid.

Alternatively, the heterocyclic compound may be selected from:

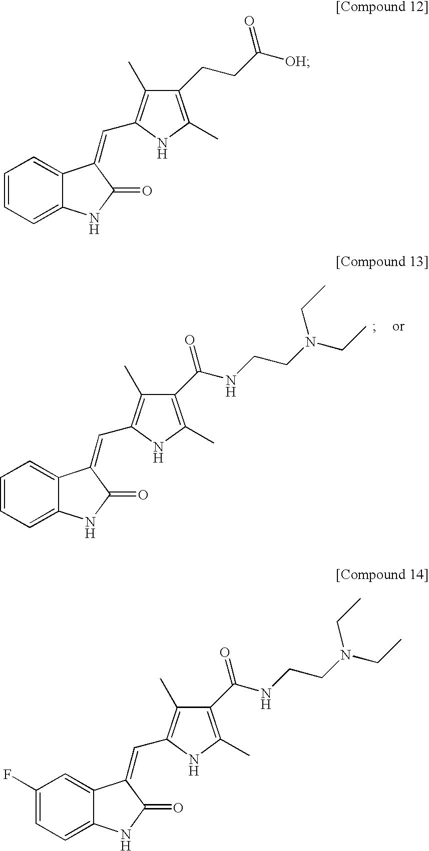

The pharmacological properties of the LRRK2 modulating agents can be evaluated, for example, in Drug Pull-Down experiments. The above-mentioned heterocyclic compounds can show activity in Drug Pull-Down experiments at concentrations below 20 μM. Compound 12 shows an IC₅₀ value of ˜1 μM.

The LRRK2 gene (SEQ ID NO: 1) may play a role in progression from mild cognitive impairment to Alzheimer's disease and progression from moderate Alzheimer's disease to more severe Alzheimer's disease. Therefore, the LRRK2 modulating agents may be able to be used to treat patients with MCI or Alzheimer's disease, to slow the progression from mild cognitive impairment to Alzheimer's disease or from moderate Alzheimer's disease to more severe Alzheimer's disease.

Identification and Characterization of Gene Sequence Variation. Due to their prevalence and widespread nature, SNPs have the potential to be important tools for locating genes that are involved in human disease conditions. See e.g., Wang et al., Science 280: 1077-1082 (1998). It is increasingly clear that the risk of developing many common disorders and the metabolism of medications used to treat these conditions are substantially influenced by underlying genomic variations, although the effects of any one variant might be small.

A SNP is said to be “allelic” in that due to the existence of the polymorphism, some members of a species may have an unmutated sequence (i.e., the original allele) whereas other members may have a mutated sequence (i.e., the variant or mutant allele).

An association between a SNP and a particular phenotype does not necessarily indicate or require that the SNP is causative of the phenotype. Instead, the association may merely be due to genome proximity between a SNP and those genetic factors actually responsible for a given phenotype, such that the SNP and said genetic factors are closely linked. That is, a SNP may be in linkage disequilibrium (“LD”) with the “true” functional variant. LD (a.k.a., allelic association) exists when alleles at two distinct locations of the genome are more highly associated than expected. Thus, a SNP may serve as a marker that has value by virtue of its proximity to a mutation that causes a particular phenotype.

In describing the polymorphic sites of the invention, reference is made to the sense strand of the gene for convenience. As recognized by the skilled artisan, however, nucleic acid molecules containing the gene may be complementary double stranded molecules and thus reference to a particular site on the sense strand refers as well to the corresponding site on the complementary antisense strand. That is, reference may be made to the same polymorphic site on either strand and an oligonucleotide may be designed to hybridize specifically to either strand at a target region containing the polymorphic site. Thus, the invention also includes single-stranded polynucleotides that are complementary to the sense strand of the genomic variants described herein.

Identification and Characterization of SNPs. Many different techniques can be used to identify and characterize SNPs, including single-strand conformation polymorphism (SSCP) analysis, heteroduplex analysis by denaturing high-performance liquid chromatography (DHPLC) and direct DNA sequencing and computational methods. Shi et al., Clin. Chem. 47:164-172 (2001). There is a wealth of sequence information in public databases.

The most common SNP-typing methods currently include hybridization, primer extension, and cleavage methods. Each of these methods must be connected to an appropriate detection system. Detection technologies include fluorescent polarization (Chan et al., Genome Res. 9:492-499 (1999)), luminometric detection of pyrophosphate release (pyrosequencing) (Ahmadiian et al., Anal. Biochem. 280:103-10 (2000)), fluorescence resonance energy transfer (FRET)-based cleavage assays, DHPLC, and mass spectrometry (Shi, Clin. Chem. 47:164-172 (2001); U.S. Pat. No. 6,300,076 Bi). Other methods of detecting and characterizing SNPs are those disclosed in U.S. Pat. Nos. 6,297,018 and 6,300,063.

Polymorphisms can also be detected using commercially available products, such as INVADER™ technology (available from Third Wave Technologies Inc. Madison, Wis., USA). In this assay, a specific upstream “invader” oligonucleotide and a partially overlapping downstream probe together form a specific structure when bound to complementary DNA template. This structure is recognized and cut at a specific site by the Cleavase enzyme, resulting in the release of the 5′ flap of the probe oligonucleotide. This fragment then serves as the “invader” oligonucleotide with respect to synthetic secondary targets and secondary fluorescently labelled signal probes contained in the reaction mixture. See also, Ryan D et al., Molecular Diagnosis 4(2): 135-144 (1999) and Lyamichev V et al., Nature Biotechnology 17: 292-296 (1999), see also U.S. Pat. Nos. 5,846,717 and 6,001,567.

The identity of polymorphisms may also be determined using a mismatch detection technique including, but not limited to, the RNase protection method using riboprobes (Winter et al., Proc. Natl. Acad. Sci. USA 82:7575 (1985); Meyers et al., Science 230:1242 (1985)) and proteins which recognize nucleotide mismatches, such as the E. coli mutS protein (Modrich P, Ann Rev Genet 25:229-253 (1991)). Alternatively, variant alleles can be identified by single strand conformation polymorphism (SSCP) analysis (Orita et al., Genomics 5:874-879 (1989); Humphries et al., in Molecular Diagnosis of Genetic Diseases, R. Elles, ed., pp. 321-340 (1996)) or denaturing gradient gel electrophoresis (DGGE) (Wartell et al., Nucl. Acids. Res. 18:2699-2706 (1990); Sheffield et al., Proc. Natl. Acad. Sci. USA 86:232-236 (1989)). A polymerase-mediated primer extension method may also be used to identify the polymorphisms. Several such methods have been described in the patent and scientific literature and include the “Genetic Bit Analysis” method (WO 92/15712) and the ligase/polymerase mediated genetic bit analysis (U.S. Pat. No. 5,679,524). Related methods are disclosed in WO 91/02087, WO 90/09455, WO 95/17676, and U.S. Pat. Nos. 5,302,509 and 5,945,283. Extended primers containing a polymorphism may be detected by mass spectrometry as described in U.S. Pat. No. 5,605,798. Another primer extension method is allele-specific PCR (Ruafio et al., Nucl. Acids. Res. 17:8392 (1989); Ruaflo et al., Nucl. Acids. Res. 19: 6877-6882 (1991); WO 93/22456; Turki et al., J. Clin. Invest. 95:1635-1641 (1995)). In addition, multiple polymorphic sites may be investigated by simultaneously amplifying multiple regions of the nucleic acid using sets of allele-specific primers as described in PCT patent application WO 89/10414.

In one embodiment, applicable to the results shown in the EXAMPLES below, blood samples from patients can be collected at the time of patient screening and DNA was extracted using, for example, the PUREGENE™ DNA Isolation Kit (D-50K). Genotyping can be performed using the TaqMan® technology or using the Third Wave Technologies Invader Assay technique.

Haplotyping and Genotyping Oligonucleotides. The invention provides methods and compositions for haplotyping and/or genotyping the gene in an individual. As used herein, the terms “genotype” and “haplotype” mean the genotype or haplotype containing the nucleotide pair or nucleotide, respectively, that is present at one or more of the polymorphic sites described herein and may optionally also include the nucleotide pair or nucleotide present at one or more additional polymorphic sites in the gene. The additional polymorphic sites may be currently known polymorphic sites or sites that are subsequently discovered.

The compositions of the invention contain oligonucleotide probes and primers designed to specifically hybridize to one or more target regions containing, or that are adjacent to, a polymorphic site. Oligonucleotide compositions of the invention are useful in methods for genotyping and/or haplotyping a gene in an individual. The methods and compositions for establishing the genotype or haplotype of an individual at the polymorphic sites described herein are useful for studying the effect of the polymorphisms in the aetiology of diseases affected by the expression and function of the protein, studying the efficacy of drugs targeting, predicting individual susceptibility to diseases affected by the expression and function of the protein and predicting individual responsiveness to drugs targeting the gene product.

Genotyping oligonucleotides of the invention may be immobilized on or synthesized on a solid surface such as a microchip, bead, or glass slide. See, e.g., WO 98/20020 and WO 98/20019.

Genotyping oligonucleotides may hybridize to a target region located one to several nucleotides downstream of one of the polymorphic sites identified herein. Such oligonucleotides are useful in polymerase-mediated primer extension methods for detecting one of the polymorphisms described herein and therefore such genotyping oligonucleotides are referred to herein as “primer-extension oligonucleotides”.

Direct Genotyping Method of the Invention. A genotyping method of the invention may involve isolating from an individual a nucleic acid mixture comprising the two copies of a gene of interest or fragment thereof, and determining the identity of the nucleotide pair at one or more of the polymorphic sites in the two copies. As will be readily understood by the skilled artisan, the two “copies” of a gene in an individual may be the same allele or may be different alleles. In a particularly preferred embodiment, the genotyping method comprises determining the identity of the nucleotide pair at each polymorphic site. Typically, the nucleic acid mixture is isolated from a biological sample taken from the individual, such as a blood sample or tissue sample. Suitable tissue samples include whole blood, semen, saliva, tears, urine, faecal material, sweat, buccal smears, skin and hair.

Direct Haplotyping Method of the Invention. A haplotyping method of the invention may include isolating from an individual a nucleic acid molecule containing only one of the two copies of a gene of interest, or a fragment thereof, and determining the identity of the nucleotide at one or more of the polymorphic sites in that copy. Direct haplotyping methods include, for example, CLASPER System™ technology (U.S. Pat. No. 5,866,404) or allele-specific long-range PCR (Michalotos-Beloin et al., Nucl. Acids. Res. 24: 4841-4843 (1996)). The nucleic acid may be isolated using any method capable of separating the two copies of the gene or fragment. As will be readily appreciated by those skilled in the art, any individual clone will only provide haplotype information on one of the two gene copies present in an individual. In one embodiment, a haplotype pair is determined for an individual by identifying the phased sequence of nucleotides at one or more of the polymorphic sites in each copy of the gene that is present in the individual. In a preferred embodiment, the haplotyping method comprises identifying the phased sequence of nucleotides at each polymorphic site in each copy of the gene.

In both the genotyping and haplotyping methods, the identity of a nucleotide (or nucleotide pair) at a polymorphic site may be determined by amplifying a target regions containing the polymorphic sites directly from one or both copies of the gene, or fragments thereof, and sequencing the amplified regions by conventional methods. The genotype or haplotype for the gene of an individual may also be determined by hybridization of a nucleic sample containing one or both copies of the gene to nucleic acid arrays and subarrays such as described in published PCT patent application WO 95/11995.

Indirect Genotyping Method using Polymorphic Sites in Linkage Disequilibrium with a Target Polymorphism. In addition, the identity of the alleles present at any of the polymorphic sites of the invention may be indirectly determined by genotyping other polymorphic sites in linkage disequilibrium with those sites of interest. As described above, two sites are said to be in linkage disequilibrium if the presence of a particular variant at one site is indicative of the presence of another variant at a second site. Stevens J C, Mol. Diag. 4: 309-317 (1999). Polymorphic sites in linkage disequilibrium with the polymorphic sites of the invention may be located in regions of the same gene or in other genomic regions.

Amplifying a Target Gene Region. The target regions may be amplified using any oligonucleotide-directed amplification method, including but not limited to polymerase chain reaction (PCR). (U.S. Pat. No. 4,965,188), ligase chain reaction (LCR) (Barany et al., Proc. Natl. Acad. Sci. USA 88:189-193 (1991); published PCT patent application WO 90/01069), and oligonucleotide ligation assay (OLA) (Landegren et al., Science 241: 1077-1080 (1988)). Oligonucleotides useful as primers or probes in such methods should specifically hybridize to a region of the nucleic acid that contains or is adjacent to the polymorphic site. Typically, the oligonucleotides are between 10 and 35 nucleotides in length and preferably, between 15 and 30 nucleotides in length. Most preferably, the oligonucleotides are 20 to 25 nucleotides long. The exact length of the oligonucleotide will depend on many factors that are routinely considered and practiced by the skilled artisan.

Other known nucleic acid amplification procedures may be used to amplify the target region including transcription-based amplification systems (U.S. Pat. No. 5,130,238; EP 329,822; U.S. Pat. No. 5,169,766, published PCT patent application WO 89/06700) and isothermal methods (Walker et al., Proc. Natl. Acad. Sci. USA 89:392-396 (1992)).

Hybridizing Allele-Specific Oligonucleotide to a Target Gene. A polymorphism in the target region may be assayed before or after amplification using one of several hybridization-based methods known in the art. Typically, allele-specific oligonucleotides are utilized in performing such methods. The allele-specific oligonucleotides may be used as differently labelled probe pairs, with one member of the pair showing a perfect match to one variant of a target sequence and the other member showing a perfect match to a different variant. In some embodiments, more than one polymorphic site may be detected at once using a set of allele-specific oligonucleotides or oligonucleotide pairs. Preferably, the members of the set have melting temperatures within 5° C., and more preferably within 2° C., of each other when hybridizing to each of the polymorphic sites being detected.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking, baking, etc. Allele-specific oligonucleotide may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibres, chips, dishes, and beads. The solid support may be treated, coated or derivatised to facilitate the immobilization of the allele-specific oligonucleotide or target nucleic acid.

Determining Population Genotypes and Haplotypes and Correlating Them with a Trait. The invention provides a method for determining the frequency of a genotype or haplotype in a population. The method comprises determining the genotype or the haplotype for a gene present in each member of the population, wherein the genotype or haplotype comprises the nucleotide pair or nucleotide detected at one or more of the polymorphic sites in the gene, and calculating the frequency at which the genotype or haplotype is found in the population. The population may be a reference population, a family population, a same sex population, a population group, or a trait population (e.g., a group of individuals exhibiting a trait of interest such as a medical condition or response to a therapeutic treatment).

In another aspect of the invention, frequency data for genotypes and/or haplotypes found in a reference population are used in a method for identifying an association between a trait and a genotype or a haplotype. The trait may be any detectable phenotype, including but not limited to susceptibility to a disease or response to a treatment. The method involves obtaining data on the frequency of the genotypes or haplotypes of interest in a reference population and comparing the data to the frequency of the genotypes or haplotypes in a population exhibiting the trait. Frequency data for one or both of the reference and trait populations may be obtained by genotyping or haplotyping each individual in the populations using one of the methods described above. The haplotypes for the trait population may be determined directly or, alternatively, by the predictive genotype to haplotype approach described above.

The frequency data for the reference and/or trait populations are obtained by accessing previously determined frequency data, which may be in written or electronic form. For example, the frequency data may be present in a database that is accessible by a computer. Once the frequency data are obtained, the frequencies of the genotypes or haplotypes of interest in the reference and trait populations are compared.

When polymorphisms are being analyzed, a calculation may be performed to correct for a significant association that might be found by chance. For statistical methods useful in the methods of the invention, see Statistical Methods in Biology, 3^{rd} edition, Bailey N T J, (Cambridge Univ. Press, 1997); Waterman M S, Introduction to Computational Biology (CRC Press, 2000) and Bioinformatics, Baxevanis A D & Ouellette B F F editors (John Wiley & Sons, Inc., 2001).

In another embodiment, the haplotype frequency data for different groups are examined to determine whether they are consistent with Hardy-Weinberg equilibrium. D. L. Hartl et al., Principles of Population Genomics, 3rd Ed. (Sinauer Associates, Sunderland, Mass., 1997).

In another embodiment, statistical analysis is performed by the use of standard ANOVA tests with a Bonferoni correction or a bootstrapping method that simulates the genotype phenotype correlation many times and calculates a significance value. ANOVA is used to test hypotheses about whether a response variable is caused by or correlates with one or more traits or variables that can be measured. L D Fisher & G vanBelle, Biostatistics: A Methodology for the Health Sciences, Ch. 10 (Wiley-Interscience, New York, 1993).

In one embodiment for predicting a haplotype pair, the analysis includes an assigning step, as follows: First, each of the possible haplotype pairs is compared to the haplotype pairs in the reference population. Generally, only one of the haplotype pairs in the reference population matches a possible haplotype pair and that pair is assigned to the individual. Occasionally, only one haplotype represented in the reference haplotype pairs is consistent with a possible haplotype pair for an individual, and in such cases the individual is assigned a haplotype pair containing this known haplotype and a new haplotype derived by subtracting the known haplotype from the possible haplotype pair.

In another embodiment, a detectable genotype or haplotype that is in linkage disequilibrium with a genotype or haplotype of interest may be used as a surrogate marker. A genotype that is in linkage disequilibrium with another genotype is indicated where a particular genotype or haplotype for a given gene is more frequent in the population that also demonstrates the potential surrogate marker genotype than in the reference population. If the frequency is statistically significant, then the marker genotype is predictive of that genotype or haplotype, and can be used as a surrogate marker.

Another method for finding correlations between haplotype content and clinical responses uses predictive models based on error-minimizing optimization algorithms, one of which is a genetic algorithm. See, R Judson, “Genetic Algorithms and Their Uses in Chemistry” in Reviews in Computational Chemistry, Ch. 10, K B Lipkowitz & D B Boyd, eds. (VCH Publishers, New York, 1997) pp. 1-73. Simulated annealing (Press et al., Numerical Recipes in C: The Art of Scientific Computing, Ch. 10 (Cambridge University Press, Cambridge, 1992), neural networks (E Rich & K Knight, Artificial Intelligence, 2nd Edition, Ch. 10 (McGraw-Hill, New York, 1991), standard gradient descent methods (Press et al., supra Ch. 10), or other global or local optimization approaches (see discussion in Judson, supra) can also be used.

Correlating Subject Genotype or Haplotype to Treatment Response. In preferred embodiments, the trait is susceptibility to a disease, severity of a disease, the staging of a disease or response to a drug. Such methods have applicability in developing diagnostic tests and therapeutic treatments for all pharmacogenetic applications where there is the potential for an association between a genotype and a treatment outcome, including efficacy measurements, pharmacokinetic measurements and side-effect measurements.

In another preferred embodiment, the trait of interest is a clinical response exhibited by a patient to some therapeutic treatment, for example, response to a drug targeting or to a therapeutic treatment for a medical condition.

To deduce a correlation between a clinical response to a treatment and a genotype or haplotype, genotype or haplotype data is obtained on the clinical responses exhibited by a population of individuals who received the treatment, hereinafter the “clinical population”. This clinical data may be obtained by analyzing the results of a clinical trial that has already been run and/or by designing and carrying out one or more new clinical trials.

The individuals included in the clinical population are usually graded for the existence of the medical condition of interest. This grading of potential patients could employ a standard physical exam or one or more lab tests. Alternatively, grading of patients could use haplotyping for situations where there is a strong correlation between haplotype pair and disease susceptibility or severity.

The therapeutic treatment of interest is administered to each individual in the trial population, and each individual's response to the treatment is measured using one or more predetermined criteria. It is contemplated that in many cases, the trial population will exhibit a range of responses and that the investigator will choose the number of responder groups (e.g., low, medium, high) made up by the various responses. In addition, the gene for each individual in the trial population is genotyped and/or haplotyped, which may be done before or after administering the treatment.

These results are then analyzed to determine if any observed variation in clinical response between polymorphism groups is statistically significant. Statistical analysis methods, which may be used, are described in L. D. Fisher & G. vanBelle, Biostatistics: A Methodology for the Health Sciences (Wiley-Interscience, New York, 1993). This analysis may also include a regression calculation of which polymorphic sites in the gene contribute most significantly to the differences in phenotype.

In one embodiment, as a first pass analysis, Fishers Exact tests are performed to evaluate response as a function of genotype.

After both the clinical and polymorphism data have been obtained, correlations between individual response and genotype or haplotype content are created. Correlations may be produced in several ways. In one method, individuals are grouped by their genotype or haplotype (or haplotype pair) (also referred to as a polymorphism group), and then the averages and standard deviations of clinical responses exhibited by the members of each polymorphism group are calculated.

From the analyses described above, the skilled artisan that predicts clinical response as a function of genotype or haplotype content may readily construct a mathematical model. The identification of an association between a clinical response and a genotype or haplotype (or haplotype pair) for the gene may be the basis for designing a diagnostic method to determine those individuals who will or will not respond to the treatment, or alternatively, will respond at a lower level and thus may require more treatment, i.e., a greater dose of a drug. The diagnostic method may take one of several forms: for example, a direct DNA test (i.e., genotyping or haplotyping one or more of the polymorphic sites in the gene), a serological test, or a physical exam measurement. The only requirement is that there be a good correlation between the diagnostic test results and the underlying genotype or haplotype. In a preferred embodiment, this diagnostic method uses the predictive haplotyping method described above.

In one embodiment, analysis is performed using a logistic remodel to take into account gender and age in addition to treatment and “high responder” (to therapeutic treatment) genotype status. In addition, an ANCOVA model can applied using the baseline value of patient response assessments as a quantitative co-variant.

Assigning a Subject to a Genotype Group. As one of skill in the art will understand, there will be a certain degree of uncertainty involved in making this determination. Therefore, the standard deviations of the control group levels would be used to make a probabilistic determination and the methods of this invention would be applicable over a wide range of probability based genotype group determinations. Thus, for example and not by way of limitation, in one embodiment, if the measured level of the gene expression product falls within 2.5 standard deviations of the mean of any of the control groups, then that individual may be assigned to that genotype group. In another embodiment if the measured level of the gene expression product falls within 2.0 standard deviations of the mean of any of the control groups then that individual may be assigned to that genotype group. In still another embodiment, if the measured level of the gene expression product falls within 1.5 standard deviations of the mean of any of the control groups then that individual may be assigned to that genotype group. In yet another embodiment, if the measured level of the gene expression product is 1.0 or less standard deviations of the mean of any of the control groups levels then that individual may be assigned to that genotype group.

Thus this process allows determination, with various degrees of probability, which group a specific subject should be placed in, and such assignment to a genotype group would then determine the risk category into which the individual should be placed.

Correlation between Clinical Response and Genotype or Haplotype. In order to deduce a correlation between clinical response to a treatment and a genotype or haplotype, it is necessary to obtain data on the clinical responses exhibited by a population of individuals who received the treatment, hereinafter the “clinical population.” This clinical data may be obtained by analyzing the results of a clinical trial that has already been run and/or the clinical data may be obtained by designing and carrying out one or more new clinical trials.

The standard control levels of the gene expression product, thus determined in the different control groups, would then be compared with the measured level of a gene expression product in a given patient. This gene expression product could be the characteristic mRNA associated with that particular genotype group or the polypeptide gene expression product of that genotype group. The patient could then be classified or assigned to a particular genotype group based on how similar the measured levels were compared to the control levels for a given group.

Computer System for Storing or Displaying Polymorphism Data. The invention also provides a computer system for storing and displaying polymorphism data determined for the gene. The computer system comprises a computer processing unit, a display, and a database containing the polymorphism data. The polymorphism data includes the polymorphisms, the genotypes and the haplotypes identified for a given gene in a reference population. In a preferred embodiment, the computer system is capable of producing a display showing haplotypes organized according to their evolutionary relationships. A computer may implement any or all analytical and mathematical operations involved in practicing the methods of the present invention. In addition, the computer may execute a program that generates views (or screens) displayed on a display device and with which the user can interact to view and analyze large amounts of information relating to the gene and its genomic variation, including chromosome location, gene structure, and gene family, gene expression data, polymorphism data, genetic sequence data, and clinical population data (e.g., data on ethnogeographic origin, clinical responses, genotypes, and haplotypes for one or more populations). The polymorphism data described herein may be stored as part of a relational database (e.g., an instance of an Oracle database or a set of ASCII flat files). These polymorphism data may be stored on the computer's hard drive or may, for example, be stored on a CD-ROM or on one or more other storage devices accessible by the computer. For example, the data may be stored on one or more databases in communication with the computer via a network.

Nucleic Acid-based Diagnostics. In another aspect, the invention provides SNP probes, which are useful in classifying subjects according to their types of genetic variation. The SNP probes according to the invention are oligonucleotides, which discriminate between SNPs in conventional allelic discrimination assays. In certain preferred embodiments, the oligonucleotides according to this aspect of the invention are complementary to one allele of the SNP nucleic acid, but not to any other allele of the SNP nucleic acid. Oligonucleotides according to this embodiment of the invention can discriminate between SNPs in various ways. For example, under stringent hybridization conditions, an oligonucleotide of appropriate length will hybridize to one SNP, but not to any other. The oligonucleotide may be labelled using a radiolabel or a fluorescent molecular tag. Alternatively, an oligonucleotide of appropriate length can be used as a primer for PCR, wherein the 3′ terminal nucleotide is complementary to one allele containing a SNP, but not to any other allele. In this embodiment, the presence or absence of amplification by PCR determines the haplotype of the SNP.

Genomic and cDNA fragments of the invention comprise at least one polymorphic site identified herein, have a length of at least 10 nucleotides, and may range up to the full length of the gene. Preferably, a fragment according to the present invention is between 100 and 3000 nucleotides in length, and more preferably between 200 and 2000 nucleotides in length, and most preferably between 500 and 1000 nucleotides in length.

Kits of the Invention. The invention provides nucleic acid and polypeptide detection kits useful for haplotyping and/or genotyping the gene in an individual. Such kits are useful for classifying individuals for the purpose of classifying individuals. Specifically, the invention encompasses kits for detecting the presence of a polypeptide or nucleic acid corresponding to a marker of the invention in a biological sample, e.g., any bodily fluid including, but not limited to, serum, plasma, lymph, cystic fluid, urine, stool, cerebrospinal fluid, ascities fluid or blood, and including biopsy samples of body tissue. For example, the kit can comprise a labelled compound or agent capable of detecting a polypeptide or an mRNA encoding a polypeptide corresponding to a marker of the invention in a biological sample and means for determining the amount of the polypeptide or mRNA in the sample, e.g., an antibody which binds the polypeptide or an oligonucleotide probe which binds to DNA or mRNA encoding the polypeptide. Kits can also include instructions for interpreting the results obtained using the kit.

In another embodiment, the invention provides a kit comprising at least two genotyping oligonucleotides packaged in separate containers. The kit may also contain other components such as hybridization buffer (where the oligonucleotides are to be used as a probe) packaged in a separate container. Alternatively, where the oligonucleotides are to be used to amplify a target region, the kit may contain, packaged in separate containers, a polymerase and a reaction buffer optimized for primer extension mediated by the polymerase, such as in the case of PCR. In a preferred embodiment, such kit may further comprise a DNA sample collecting means.

For antibody-based kits, the kit can comprise, e.g., (1) a first antibody, e.g., attached to a solid support, which binds to a polypeptide corresponding to a marker or the invention; and, optionally (2) a second, different antibody which binds to either the polypeptide or the first antibody and is conjugated to a detectable label.

For oligonucleotide-based kits, the kit can comprise, e.g., (1) an oligonucleotide, e.g., a detectably-labelled oligonucleotide, which hybridizes to a nucleic acid sequence encoding a polypeptide corresponding to a marker of the invention; or (2) a pair of primers useful for amplifying a nucleic acid molecule corresponding to a marker of the invention.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit.

Nucleic Acid Sequences of the Invention. In one aspect, the invention comprises one or more isolated polynucleotides. The invention also encompasses allelic variants of the same, that is, naturally occurring alternative forms of the isolated polynucleotides that encode mutant polypeptides that are identical, homologous or related to those encoded by the polynucleotides. Alternatively, non-naturally occurring variants may be produced by mutagenesis techniques or by direct synthesis techniques well-known in the art.

Accordingly, nucleic acid sequences capable of hybridizing at low stringency with any nucleic acid sequences encoding mutant polypeptide of the present invention are considered to be within the scope of the invention. Standard stringency conditions are well characterized in standard molecular biology cloning texts. See, for example Molecular Cloning A Laboratory Manual, 2nd Ed., ed., Sambrook, Fritsch, & Maniatis (Cold Spring Harbor Laboratory Press, 1989); DNA Cloning, Volumes I and II, D. N. Glover, ed. (1985); Oligonucleotide Synthesis, M. J. Gait, ed. (1984); Nucleic Acid Hybridization, B. D. Hames & S. J. Higgins, eds (1984).

Characterizing Gene Expression Level. Methods to detect and measure mRNA levels (i.e., gene transcription level) and levels of polypeptide gene expression products (i.e., gene translation level) are well-known in the art and include the use of nucleotide microarrays and polypeptide detection methods involving mass spectrometers and/or antibody detection and quantification techniques. See also, Tom Strachan & Andrew Read, Human Molecular Genetics, 2^{nd} Edition. (John Wiley and Sons, Inc. Publication, New York, 1999)).

Determination of Target Gene Transcription. The determination of the level of the expression product of the gene in a biological sample, e.g., the tissue or body fluids of an individual, may be performed in a variety of ways. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells. See, e.g., Ausubel et al., Ed., Curr. Prot. Mol. Biol. (John Wiley & Sons, New York, 1987-1999).

In one embodiment, the level of the mRNA expression product of the target gene is determined. Methods to measure the level of a specific mRNA are well-known in the art and include Northern blot analysis, reverse transcription PCR and real time quantitative PCR or by hybridization to a oligonucleotide array or microarray. In other more preferred embodiments, the determination of the level of expression may be performed by determination of the level of the protein or polypeptide expression product of the gene in body fluids or tissue samples including but not limited to blood or serum. Large numbers of tissue samples can readily be processed using techniques well-known to those of skill in the art, such as, e.g., the single-step RNA isolation process of U.S. Pat. No. 4,843,155.

The isolated mRNA can be used in hybridization or amplification assays that include, but are not limited to, Southern or Northern analyses, PCR analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, e.g., a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a marker of the present invention. Other suitable probes for use in the diagnostic assays of the invention are described herein. Hybridization of an mRNA with the probe indicates that the marker in question is being expressed.

In one format, the probes are immobilized on a solid surface and the mRNA is contacted with the probes, for example, in an Affymetrix gene chip array (Affymetrix, Calif. USA). A skilled artisan can readily adapt known mRNA detection methods for use in detecting the level of mRNA encoded by the markers of the present invention.

An alternative method for determining the level of mRNA corresponding to a marker of the present invention in a sample involves the process of nucleic acid amplification, e.g., by RT-PCR (the experimental embodiment set forth in U.S. Pat. No. 4,683,202); ligase chain reaction (Barany et al, Proc. Natl. Acad Sci. USA 88:189-193 (1991)) self-sustained sequence replication (Guatelli et al., Proc. Natl. Acad. Sci. USA 87: 1874-1878 (1990)); transcriptional amplification system (Kwoh et al., Proc. Natl. Acad. Sci. USA 86: 1173-1177 (1989)); Q-Beta Replicase (Lizardi et al., Biol. Technology 6: 1197 (1988)); rolling circle replication (U.S. Pat. No. 5,854,033); or any other nucleic acid amplification method, followed by the detection of the amplified molecules using techniques well-known to those of skill in the art. These detection schemes are especially useful for the detection of the nucleic acid molecules if such molecules are present in very low numbers. As used herein, “amplification primers” are defined as being a pair of nucleic acid molecules that can anneal to 5′ or 3′ regions of a gene (plus and minus strands, respectively, or vice-versa) and contain a short region in between. In general, amplification primers are from about 10-30 nucleotides in length and flank a region from about 50-200 nucleotides in length.

Real-time quantitative PCR (RT-PCR) is one way to assess gene expression levels, e.g., of genes of the invention, e.g., those containing SNPs and polymorphisms of interest. The RT-PCR assay utilizes an RNA reverse transcriptase to catalyze the synthesis of a DNA strand from an RNA strand, including an mRNA strand. The resultant DNA may be specifically detected and quantified and this process may be used to determine the levels of specific species of mRNA. One method for doing this is TAQMAN® (PE Applied Biosystems, Foster City, Calif., USA) and exploits the 5′ nuclease activity of AMPLITAQ GOLD™ DNA polymerase to cleave a specific form of probe during a PCR reaction. This is referred to as a TAQMAN™ probe. See Luthra et al., Am. J. Pathol. 153: 63-68 (1998); Kuimelis et al., Nucl. Acids Symp. Ser. 37: 255-256 (1997); and Mullah et al., Nucl. Acids Res. 26(4): 1026-1031 (1998)). During the reaction, cleavage of the probe separates a reporter dye and a quencher dye, resulting in increased fluorescence of the reporter. The accumulation of PCR products is detected directly by monitoring the increase in fluorescence of the reporter dye. Heid et al., Genome Res. 6(6): 986-994 (1996)). The higher the starting copy number of nucleic acid target, the sooner a significant increase in fluorescence is observed. See Gibson, Heid & Williams et al., Genome Res. 6: 995-1001 (1996).

Other technologies for measuring the transcriptional state of a cell produce pools of restriction fragments of limited complexity for electrophoretic analysis, such as methods combining double restriction enzyme digestion with phasing primers (see, e.g., EP 0 534858 A1), or methods selecting restriction fragments with sites closest to a defined mRNA end. (See, e.g., Prashar & Weissman, Proc. Natl. Acad. Sci. USA 93(2) 659-663 (1996)).

Other methods statistically sample cDNA pools, such as by sequencing sufficient bases, e.g., 20-50 bases, in each of multiple cDNAs to identify each cDNA, or by sequencing short tags, e.g., 9-10 bases, which are generated at known positions relative to a defined mRNA end pathway pattern. See, e.g., Velculescu, Science 270: 484-487 (1995). The cDNA levels in the samples are quantified and the mean, average and standard deviation of each cDNA is determined using by standard statistical means well-known to those of skill in the art. Norman T. J. Bailey, Statistical Methods In Biology, 3rd Edition (Cambridge University Press, 1995).

Detection of Polypeptides. Immunological Detection Methods. Expression of the protein encoded by the genes of the invention can be detected by a probe which is detectably labelled, or which can be subsequently labelled. The term “labelled”, with regard to the probe or antibody, is intended to encompass direct-labelling of the probe or antibody by coupling, i.e., physically linking, a detectable substance to the probe or antibody, as well as indirect-labelling of the probe or antibody by reactivity with another reagent that is directly-labelled. Examples of indirect labelling include detection of a primary antibody using a fluorescently-labelled secondary antibody and end-labelling of a DNA probe with biotin such that it can be detected with fluorescently-labelled streptavidin. Generally, the probe is an antibody that recognizes the expressed protein. A variety of formats can be employed to determine whether a sample contains a target protein that binds to a given antibody. Immunoassay methods useful in the detection of target polypeptides of the present invention include, but are not limited to, e.g., dot blotting, western blotting, protein chips, competitive and non-competitive protein binding assays, enzyme-linked immunosorbant assays (ELISA), immunohistochemistry, fluorescence activated cell sorting (FACS), and others commonly used and widely-described in scientific and patent literature, and many employed commercially. A skilled artisan can readily adapt known protein/antibody detection methods for use in determining whether cells express a marker of the present invention and the relative concentration of that specific polypeptide expression product in blood or other body tissues. Proteins from individuals can be isolated using techniques that are well-known to those of skill in the art. The protein isolation methods employed can, e.g., be such as those described in Harlow & Lane, Antibodies: A Laboratory Manual (Cold Spring Harbor Laboratory Press, Cold Spring Harbor, N.Y., 1988)).

For the production of antibodies to a protein encoded by one of the disclosed genes, various host animals may be immunized by injection with the polypeptide, or a portion thereof. Such host animals may include, but are not limited to, rabbits, mice and rats. Various adjuvants may be used to increase the immunological response, depending on the host species including, but not limited to, Freund's (complete and incomplete), mineral gels, such as aluminium hydroxide; surface active substances, such as lysolecithin, pluronic polyols, polyanions, peptides, oil emulsions, keyhole limpet hemocyanin and dinitrophenol; and potentially useful human adjuvants, such as bacille Camette-Guerin (BCG) and Corynebacterium parvum.

Monoclonal antibodies (mAbs), which are homogeneous populations of antibodies to a particular antigen, may be obtained by any technique that provides for the production of antibody molecules by continuous cell lines in culture. These include, but are not limited to, the hybridoma technique of Kohler & Milstein, Nature 256: 495-497 (1975); and U.S. Pat. No. 4,376,110; the human B-cell hybridoma technique of Kosbor et al., Immunol. Today 4: 72 (1983); Cole et al., Proc. Natl. Acad. Sci. USA 80: 2026-2030 (1983); and the EBV-hybridoma technique of Cole et al., Monoclonal Antibodies and Cancer Therapy (Alan R. Liss, Inc., 1985) pp. 77-96.

In addition, techniques developed for the production of “chimeric antibodies” (see Morrison et al, Proc. Natl. Acad. Sci. USA 81: 6851-6855 (1984); Neuberger et al., Nature 312: 604-608 (1984); and Takeda et al., Nature 314: 452454 (1985)), by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived form a murine mAb and a human immunoglobulin constant region.

Alternatively, techniques described for the production of single chain antibodies (U.S. Pat. No. 4,946,778; Bird, Science 242: 423-426 (1988); Huston et al., Proc. Natl. Acad. Sci. USA 85: 5879-5883 (1988); and Ward et al., Nature 334: 544-546 (1989)) can be adapted to produce differentially expressed gene single-chain antibodies.

Techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibodies or antibody fragments can be used in methods, such as Western blots or immunofluorescence techniques, to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros and magnetite.

A useful method, for ease of detection, is the sandwich ELISA, of which a number of variations exist, all of which are intended to be used in the methods and assays of the present invention. As used herein, “sandwich assay” is intended to encompass all variations on the basic two-site technique. Immunofluorescence and EIA techniques are both very well-established in the art. However, other reporter molecules, such as radioisotopes, chemiluminescent or bioluminescent molecules may also be employed. It will be readily apparent to the skilled artisan how to vary the procedure to suit the required use.

Whole genome monitoring of protein, i.e., the “proteome,” can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to testing or confirming a biological network model of interest. As noted above, methods for making monoclonal antibodies are well-known. See, e.g., Harlow & Lane, Antibodies: A Laboratory Manuar” (Cold Spring Harbor Laboratory Press, Cold Spring Harbor, New York, 1988)). In a preferred embodiment, monoclonal antibodies are raised against synthetic peptide fragments designed based on genomic sequence of the cell. With such an antibody array, proteins from the cell are contacted to the array and their binding is measured with assays known in the art.

Detection of Polypeptides. Two-Dimensional Gel Electrophoresis. Two-dimensional gel electrophoresis is well-known in the art and typically involves isoelectric focusing along a first dimension followed by SDS-PAGE electrophoresis along a second dimension. See, e.g., Hames et al., Gel Electrophoresis of Proteins: A Practical Approach (IRL Press, New York, 1990); Shevchenko et al., Proc. Natl. Acad. Sci. USA 93: 14440-14445 (1996); Sagliocco et al., Yeast 12: 1519-1533 (1996); and Lander, Science 274: 536-539 (1996).

Detection of Polypeptides. Mass Spectroscopy. The identity as well as expression level of target polypeptide can be determined using mass spectrocopy technique (MS). MS-based analysis methodology is useful for analysis of isolated target polypeptide as well as analysis of target polypeptide in a biological sample. MS formats for use in analyzing a target polypeptide include ionization (I) techniques, such as, but not limited to, matrix assisted laser desorption (MALDI), continuous or pulsed electrospray ionization (ESI) and related methods, such as ionspray or thermospray, and massive cluster impact (MCI). Such ion sources can be matched with detection formats, including linear or non-linear reflectron time of flight (TOF), single or multiple quadrupole, single or multiple magnetic sector, Fourier transform ion cyclotron resonance (FTICR), ion trap and combinations thereof such as ion-trap/TOF. For ionization, numerous matrix/wavelength combinations (e.g., matrix assisted laser desorption (MALDI)) or solvent combinations (e.g., ESI) can be employed.

For mass spectroscopy (MS) analysis, the target polypeptide can be solubilised in an appropriate solution or reagent system. The selection of a solution or reagent system, e.g., an organic or inorganic solvent, will depend on the properties of the target polypeptide and the type of MS performed, and is based on methods well-known in the art. See, e.g. Vorm et al., Anal. Chem. 61: 3281 (1994) for MALDI; and Valaskovic et al., Anal. Chem. 67: 3802 (1995), for ESI. MS of peptides also is described, e.g., in International PCT Application No. WO 93/24834 and U.S. Pat. No. 5,792,664. A solvent is selected that minimizes the risk that the target polypeptide will be decomposed by the energy introduced for the vaporization process. A reduced risk of target polypeptide decomposition can be achieved, e.g., by embedding the sample in a matrix. A suitable matrix can be an organic compound such as a sugar, e.g., a pentose or hexose, or a polysaccharide such as cellulose. Such compounds are decomposed thermolytically into CO₂ and H₂O such that no residues are formed that can lead to chemical reactions. The matrix also can be an inorganic compound, such as nitrate of ammonium, which is decomposed essentially without leaving any residue. Use of these and other solvents is known to those of skill in the art. See, e.g., U.S. Pat. No. 5,062,935. Electrospray MS has been described by Fenn et al., J. Phys. Chem. 88: 4451-4459 (1984); and PCT Application No. WO 90/14148; and current applications are summarized in review articles. See Smith et al., Anal. Chem. 62: 882-89 (1990); and Ardrey, Spectroscopy 4: 10-18 (1992).

The mass of a target polypeptide determined by MS can be compared to the mass of a corresponding known polypeptide. For example, where the target polypeptide is a mutant protein, the corresponding known polypeptide can be the corresponding non-mutant protein, e.g., wild-type protein. With ESI, the determination of molecular weights in femtomole amounts of sample is very accurate due to the presence of multiple ion peaks, all of which can be used for mass calculation. Sub-attomole levels of protein have been detected, e.g., using ESI MS (Valaskovic et al., Science 273: 1199-1202 (1996)) and MALDI MS (Li et al., J. Am. Chem. Soc. 118: 1662-1663 (1996)).

Matrix Assisted Laser Desorption (MALDI). The level of the target protein in a biological sample, e.g., body fluid or tissue sample, may be measured by means of mass spectrometric (MS) methods including, but not limited to, those techniques known in the art as matrix-assisted laser desorption/ionization, time-of-flight mass spectrometry (MALDI-TOF-MS) and surfaces enhanced for laser desorption/ionization, time-of-flight mass spectrometry (SELDI-TOF-MS) as further detailed below. Methods for performing MALDI are well-known to those of skill in the art. See, e.g., Juhasz et al., Analysis, Anal. Chem. 68: 941-946 (1996), and see also, e.g., U.S. Pat. Nos. 5,777,325; 5,742,049; 5,654,545; 5,641,959; 5,654,545 and 5,760,393 for descriptions of MALDI and delayed extraction protocols. Numerous methods for improving resolution are also known. MALDI-TOF-MS has been described by Hillenkamp et al., Biological Mass Spectrometry, Burlingame & McCloskey, eds. (Elsevier Science Publ., Amsterdam, 1990) pp. 49-60.

A variety of techniques for marker detection using mass spectroscopy can be used. See Bordeaux Mass Spectrometry Conference Report, Hillenkamp, Ed., pp. 354-362 (1988); Bordeaux Mass Spectrometry Conference Report, Karas & Hillenkamp, eds., pp. 416-417 (1988); Karas & Hillenkamp, Anal. Chem. 60: 2299-2301 (1988); and Karas et al., Biomed. Environ. Mass Spectrum 18: 841-843 (1989). The use of laser beams in TOF-MS is shown, e.g., in U.S. Pat. Nos. 4,694,167; 4,686,366, 4,295,046 and 5,045,694, which are incorporated herein by reference in their entireties. Other MS techniques allow the successful volatilization of high molecular weight biopolymers, without fragmentation, and have enabled a wide variety of biological macromolecules to be analyzed by mass spectrometry.

Surfaces Enhanced for Laser Desorption/Ionization (SELDI). Other techniques are used which employ new MS probe element compositions with surfaces that allow the probe element to actively participate in the capture and docking of specific analytes, described as Affinity Mass Spectrometry (AMS). See SELDI patents U.S. Pat. Nos. 5,719,060; 5,894,063; 6,020,208; 6,027,942; 6,124,137; and U.S. Patent application No. U.S. 2003/0003465. Several types of new MS probe elements have been designed with Surfaces Enhanced for Affinity Capture (SEAC). See Hutchens & Yip, Rapid Commun. Mass Spectrom. 7: 576-580 (1993). SEAC probe elements have been used successfully to retrieve and tether different classes of biopolymers, particularly proteins, by exploiting what is known about protein surface structures and biospecific molecular recognition. The immobilized affinity capture devices on the MS probe element surface, i.e., SEAC, determines the location and affinity (specificity) of the analyte for the probe surface, therefore the subsequent analytical MS process is efficient.

Within the general category of SELDI are three separate subcategories: (1) Surfaces Enhanced for Neat Desorption (SEND), where the probe element surfaces, i.e., sample presenting means, are designed to contain Energy Absorbing Molecules (EAM) instead of “matrix” to facilitate desorption/ionizations of analytes added directly (neat) to the surface. (2) SEAC, where the probe element surfaces, i.e., sample presenting means, are designed to contain chemically defined and/or biologically defined affinity capture devices to facilitate either the specific or non-specific attachment or adsorption (so-called docking or tethering) of analytes to the probe surface, by a variety of mechanisms (mostly non-covalent). (3) Surfaces Enhanced for Photolabile Attachment and Release (SEPAR), where the probe element surfaces, i.e., sample presenting means, are designed or modified to contain one or more types of chemically defined cross-linking molecules to serve as covalent docking devices. The chemical specificities determining the type and number of the photolabile molecule attachment points between the SEPAR sample presenting means (i.e., probe element surface) and the analyte (e.g., protein) may involve any one or more of a number of different residues or chemical structures in the analyte (e.g., His, Lys, Arg, Tyr, Phe and Cys residues in the case of proteins and peptides).

Other Aspects of the Biological State. In various embodiments of the invention, aspects of the biological activity state, or mixed aspects can be measured in order to obtain drug and pathway responses. The activities of proteins relevant to the characterization of cell function can be measured, and embodiments of this invention can be based on such measurements. Activity measurements can be performed by any functional, biochemical or physical means appropriate to the particular activity being characterized. Where the activity involves a chemical transformation, the cellular protein can be contacted with natural substrates, and the rate of transformation measured. Where the activity involves association in multimeric units, e.g., association of an activated DNA binding complex with DNA, the amount of associated protein or secondary consequences of the association, such as amounts of mRNA transcribed, can be measured. Also, where only a functional activity is known, e.g., as in cell cycle control, performance of the function can be observed. However known and measured, the changes in protein activities form the response data analyzed by the methods of this invention. In alternative and non-limiting embodiments, response data may be formed of mixed aspects of the biological state of a cell. Response data can be constructed from, e.g., changes in certain mRNA abundances, changes in certain protein abundances and changes in certain protein activities.

The following EXAMPLES are presented in order to more fully illustrate the preferred embodiments of the invention. These EXAMPLE should in no way be construed as limiting the scope of the invention, as defined by the appended claims.

### Example 1

Association of the Common Polymorphisms in the LRRK2 Gene with Progression of Alzheimer's Disease (AD)

The objective of this EXAMPLE was to test whether variations in the LRRK2 gene are associated with the progression to Alzheimer's disease (AD) in subjects with mild cognitive impairment (MCI).

We tested for 2 common polymorphisms of the LRRK2 gene: T1602S and T2352M. For the LRRK2 gene (SEQ ID NO: 1), pairwise Linkage Disequilibrium (LD) analysis showed that T1602S and T2352M are in strong LD (D′=0.979). For the T1602S mutation, the allele frequency (for the minor allele) was found to be as follows for the patient populations: PD=27%, AD=28%, MCI=29%, ALS=31%.

THR1602SER mutation. Progression to Alzheimer's disease data of a 3-4 year study in 537 subjects was used to investigate the effect of the two LRRK2 common polymorphisms on the likelihood of progression to AD. The Investigation Into Delay to Diagnosis of Alzheimer's Disease With Exelon (InDDex) study was a placebo-controlled, 4-year longitudinal study to evaluate efficacy of Exelon® in the individuals with mild cognitive impairment (MCI). The clinical trial followed patients suffering from mild cognitive impairment and given either Exelon® (rivastigmine) at various doses or placebo and followed their conversion to Alzheimer's disease (AD). Feldman H et al., Neurology 62:1199-1201 (2004). The trial had an optional DNA collection component.

In the InDDEx (MCI) study, we found that the TT genotype (or Thr/Thr) of the polymorphism T1602S was significantly associated with higher rate of progression to Alzheimer's disease (TABLE 1).

TABLE 1
Rate of conversion from MCI to AD by T1602S genotype

| Conver- | LRRK2 genotype (116028) | | | | | |
|---|---|---|---|---|---|---|
| sion | Thr/Thr | Thr/Ser | Ser/Ser | Hazard | 95% | P |
| to AD | (n = 38) | (n = 174) | (n = 217) | ratio* | CI | value** |
| Yes, % | 34.21 | 14.94 | 17.05 | 3.009 | (1.63, | 0.0021 |
| No, % | 65.79 | 85.06 | 82.95 | | 5.56) | |

*Cox proportional hazards. Age, gender and years of education were included in the model.
**Log-rank test for the time to conversion.

Similar to the APOE-E4 allele, in the presence of a BuChE-K variant, LRRK2 polymorphism T1602S showed a greater association with the rate of conversion from mild cognitive impairment to Alzheimer's disease.

To verify these findings, we further tested the correlation between this common LRRK2 polymorphism and cognitive performance over 6 months in the 178 placebo-treated AD patients enrolled in the IDEAL study. In the IDEAL (AD) study, LRRK2 polymorphism T1602S showed a same trend of the association observed in the MCI study. The Alzheimer's disease patients with TT genotype of T1602S tended to decline faster on cognitive performance over 6 months, especially in the presence of a BuChE-K variant.

THR2352MET mutation. In addition, in the InDDeX study, the CC genotype (or Thr/Thr) of T2352 showed a trend of associated with higher rate of conversion from mild cognitive impairment to Alzheimer's disease (TABLE 2).

TABLE 2
Rate of conversion from MCI to AD by T2352M genotype

| Conver- | LRRK2 genotype (T2352M) | | | | | |
|---|---|---|---|---|---|---|
| sion | Met/Met | Thr/Met | Thr/Thr | Hazard | 95% | P |
| to AD | (n = 60) | (n = 196) | (n = 188) | ratio* | CI | value** |
| Yes, % | 16.67 | 15.31 | 21.81 | 1.436 | (0.93, | 0.0823 |
| No, % | 83.33 | 84.69 | 78.19 | | 2.22) | |

*Cox proportional hazards. Age, gender and years of education were included in the model as covariants.
**Log-rank test for the time to conversion.

To verify these findings, we further tested the correlation between the two common LRRK2 polymorphisms (see above) and cognitive performance over 6 months in the 178 placebo-treated AD patients enrolled in the IDEAL study. In the IDEAL (AD) study, LRRK2 polymorphisms T1602S and T2352 both showed a same trend of the association observed in the MCI study. The Alzheimer's disease patients with CC genotype of LRRK2-T2352 tended to decline faster on cognitive performance over 6 months, especially in the presence of a BuChE-K variant.

Thus, common polymorphisms in the LRRK2 gene influence the rate of progression to Alzheimer's disease in subjects with mild cognitive impairment, suggesting that LRRK2 affects Alzheimer's disease pathogenesis.

### Example 2

### Analysis of Genetic Variations of LRRK2

GLY2019SER mutation. In a screen of patients, with results confirmed by re-sequencing, we found the following: For Parkinson's disease (PD): 6 out of 483 patients carry the G2019S mutation (1.24%). For Parkinson's disease with dementia (PDD): 1 out of 391 patients carry the G2019S mutation (0.26%). For Alzheimer's disease (AD): None of the 373 patients carry the G2019S mutation. For Mild cognitive impairment (MCI): None of the 448 patients carry the G2019S mutation. For Amyotrophic lateral sclerosis (ALS): None of the 483 patients carry the G2019S mutation.

Only 4 of the 6 subjects carrying the G2019S mutation had clinical data. All 4 are male Caucasians. Progress was relatively faster (mutant vs wild-type, for 26 weeks), with the following results: UPDRSII: 1 vs 0.27. UPDRSIII: 4 vs −0.15.

We concluded that: Approximately 1.24% sporadic late-onset cases carry the mutation, which is similar to the frequency reported in the literature. Only about 0.26% PDD cases carry the G2019S mutation. This mutation is not common in AD, MCI and ALS. The mutation may be correlated with faster decline of motor function

### Equivalents

The details of one or more embodiments of the invention are set forth in the accompanying description above. Although any methods and materials similar or equivalent to those described herein can be used in the practice or testing of the present invention, the preferred methods and materials are now described. Other features, objects, and advantages of the invention will be apparent from the description and the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All references cited herein are incorporated herein by reference in their entirety and for all purposes to the same extent as if each individual publication, patent, or patent application was specifically and individually indicated to be incorporated by reference in its entirety for all purposes.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled.

We claim:

1. Use of a LRRK2 modulating agent in the manufacture of a medicament for the treatment of Alzheimer's disease a selected patient population, wherein the patient population is selected on the basis of polymorphisms in the leucine-rich repeat kinase 2 (LRRK2) gene that are indicative of progression from mild cognitive impairment (MCI) to Alzheimer's disease.

2. The use of claim 1, wherein the LRRK2 modulating agent is a heterocyclic compound.

3. The use of claim 1, wherein the treatment of Alzheimer's disease slows the progression by the patient from mild cognitive impairment (MCI) to Alzheimer's disease.

4. The use of claim 1, wherein the treatment of Alzheimer's disease slows the progression by the patient from moderate Alzheimer's disease to severe Alzheimer's disease.

5. The use of claim 1, wherein the polymorphism in the LRRK2 gene is selected from the group consisting T1602S and T2352.

6. The use of claim 5, wherein the T1602S locus of the patient has a TT (Thr/Thr) genotype.

7. The use of claim 5, wherein T2352 locus of the patient has a CC (Thr/Thr) genotype.

8. A method for predicting the progression of Alzheimer's disease in a subject, comprising the steps of:
(a) obtaining a tissue sample from a subject;
(b) assaying the sample for the presence of a genetic polymorphism indicative of progression of the subject from mild cognitive impairment (MCI) to Alzheimer's disease; wherein the presence of a genetic polymorphism indicative of progression of the subject from mild cognitive impairment to Alzheimer's disease in the subject predicts that the subject is at increased risk for progression from mild cognitive impairment to Alzheimer's disease.

9. The method of claim 8, wherein the tissue sample is a blood sample.

10. The method of claim 8, wherein the genetic polymorphism is selected from the group consisting T1602S and T2352.

11. The method of claim 8, further comprising the step of.
(c) if the subject is predicted to have a genetic polymorphism indicative of progression of the subject from mild cognitive impairment to Alzheimer's disease, then administering to the subject a LRRK2 modulating agent to slow the progression from mild cognitive impairment to Alzheimer's disease or from moderate Alzheimer's disease to severe Alzheimer's disease.
